(19) United States
(12) Patent Application Publication
(10) Pub. No.: US 2014/0051092 A1
(43) Pub. Date: Feb. 20, 2014

(54) Method And Apparatus For The Analysis Of Biological Samples

(71) Applicants: Jonathan Paul Williams, Cimla (GB); Christopher John Hughes, Manchester (GB); Johannes Petrus Cornelis Vissers, Netherlands (NL)

(72) Inventors: Jonathan Paul Williams, Cimla (GB); Christopher John Hughes, Manchester (GB); Johannes Petrus Cornelis Vissers, Netherlands (NL)

(73) Assignee: MICROMASS UK LIMITED, Manchester (GB)

(21) Appl. No.: 14/110,258

(22) PCT Filed: Apr. 12, 2012
(86) PCT No.: PCT/GB2012/050807

§ 371 Date: Oct. 29, 2013

Related U.S. Application Data
(60) Provisional application No. 61/476,873, filed on Apr. 19, 2011.

(30) Foreign Application Priority Data
Apr. 15, 2011 (GB) 1106456.5
Jun. 7, 2011 (GB) 1109469.5

(51) Int. Cl. G01N 33/72

(52) U.S. Cl. CPC G01N 33/721
USPC 435/7.1; 435/287.1

(57) ABSTRACT

A method for the detection and quantitation of analytes of interest and variants of the analyte of interest comprising the steps of:
(i) providing a sample containing an analyte of interest;
(ii) spiking sample with known amount of calibrant;
(iii) performing an LCMS or LCMSMS analysis on the spiked sample to produce a data set;
(iv) determining from said data set the relative quantity of analyte of interest to calibrant;
(v) calculating the absolute quantity of the analyte of interest from said relative quantity of analyte of interest and said known amount of calibrant;
(vi) searching for one or more previously identified candidate sequences for one or more known variants denoted by one or more specific peaks to identify the presence of said one or more known variants within the sample;
(vii) determining the relative quantity or amount of said one or more variants to said analyte of interest; and
(viii) calculating the absolute quantity or amount of any of said one or more variants present in the sample.

This invention relates generally to the analysis of biological samples and more specifically, to methods and apparatus for the identification and quantification of biological species and their variants in samples.

Liquid Chromatography (LC) and Mass Spectrometry (MS) techniques are known ways to analyse samples, in order to identify and quantify individual elements within a sample. It is often desirable to analyse biological samples to determine the presence and/or quantity of any constituent of interest within a biological sample using LC and/or MS instruments.

The use of these LC and/or MS instruments for the purpose of determining the presence and/or quantity of constituents within a biological sample can be very useful in order to identify potential illnesses or deficiencies that may be present in the patient.

Variants to the constituents of interest in the biological samples may also be present in the samples. These variants would not usually be identified to be different to the species by an analysis, and so may contribute to the levels of the constituents that are measured within the sample. In some cases, this may give inaccurate results, which may lead to incorrect reflections on the state of health of the patient.

Silva et al (Silva J C, Gorenstein M V, Li G Z, Vissers J P, Geromanos S J, Mol CellProteomics, 2006 Jan; 5(1):144-156) discloses a method of determining the concentration of proteins by an LCMS method. However, this method gives a level of total protein concentration, without identifying the level of any variants present in the sample.

Green et al disclose a method if identifying protein variants by mass spectrometric methods. However, these methods are only related to the identification of variants in a sample, and not to the quantification of the variant nor the total protein concentration in the sample.

One potential analyte of interest is Hemoglobin. Hospitals typically use a haematology automated analyser to measure the concentration of Hb in Blood (For example, the SYSMEX XE-2100). Here Hb determination is achieved using the sodium lauryl sulfate (SLS)-hemoglobin method and fluoresecent flow cytometry. However, this method also cannot determine the concentration of individual variants but only provide a total concentration value.

In, for example a pregnant mother, it would be advantageous to identify the levels of hemoglobin present in a sample, and at the same time, to flag any variants that may be present in the sample so that any further potential health issues that variants in the subject's hemoglobin levels may lead to. In the example of the pregnant mother, this may include identifying the sickle variant, and upon discovery of this variant testing the father for the same variant, in order to identify potential health problems for the child.

Using known techniques to perform this analysis does not enable a user to be able to derive all the useful information from a sample. Therefore, it is desirable to be able to provide a method of identifying and quantifying the amounts of a biological sample of interest and any variants present in the sample in a single test.

The present invention provides methods and apparatus that are particularly suited for identification and quantification of analytes of interest within biological samples and any variants to the analytes of interest within those samples. More specifically, the methods and apparatus of the present invention enable more accurate identification of potentially harmful variants within a sample to enable better characterisation of potential defects in the sample in analysis.

One aspect of the invention provides a method for the detection and quantitation of analytes of interest and variants of the analyte of interest comprising the steps of (i) providing a sample containing an analyte of interest, (ii) spiking the sample with a known amount of calibrant, (iii) performing an LCMS or LCMSMS analysis on the spiked sample to produce a data set, (iv) determining from said data set the relative quantity of analyte of interest to calibrant, (v) calculating the absolute quantity of the analyte of interest from said relative quantity of analyte of interest and said known amount of calibrant, (vi) searching for one or more previously identified candidate sequences for one or more known variants denoted by one or more specific peaks to identify the presence of said one or more known variants within the sample, (vii) determining the relative quantity or amount of said one or more variants to said analyte of interest and (viii) calculating the absolute quantity or amount of any of said one or more variants present in the sample.

The method may further comprise identifying the candidate sequences, for example before the searching step, which identification step may comprise spiking a sample with a known amount of calibrant and/or performing an LCMS or LCMSMS analysis on the spiked sample to produce a candidate data set and/or selecting from the candidate data set one or more sequences for data normalisation and/or scaling the sample intensities to sequences of interest and/or identifying one or more candidate sequences that can he used for correction.

A further aspect of the invention provides a method for the identification of candidate sequences for one or more known variants of an analyte, the method comprising the steps of (i) spiking a sample, e.g. a known sample, with a known amount of calibrant; (ii) performing an LCMS or LCMSMS analysis on the spiked sample to produce a candidate data set (iii) selecting from the candidate data set one or more sequences for data normalisation; (iv) scaling the sample intensities to sequences of interest and (v) identifying one or more candidate sequences that can be used for correction.

In the preferred embodiment, the mass spectrometer has a Quadrupole OAToF geometry.

In the most preferred embodiment, the mass spectrometer is arranged to switch between a high and a low fragmentation mode

In one preferred embodiment a digest may be added to said sample. Additionally, or alternatively, denaturation of said sample may be performed

In one embodiment the analyte of interest is hemoglobin.

A further aspect of the invention provides a system for carrying out a method as described above, the system comprising: a mass spectrometer for producing at least one measured spectrum of data from a sample and a processor configured or programmed or adapted to carry out a method as described above.

In some embodiments, the system further comprises a memory means for storing a library of candidate sequences.

A further aspect of the invention provides a computer program element, for example comprising computer readable program code means, e.g. for causing a processor to execute a procedure to implement the method described above.

The computer program element may be embodied on a computer readable medium.

A further aspect of the invention provides a computer readable medium having a program stored thereon, for example where the program is to make a computer execute a procedure, e.g. to implement the method described above.

A further aspect of the invention provides a mass spectrometer suitable for carrying out, or specifically adapted to carry out, a method as described above and/or comprising a program element as described above a computer readable medium as described above.

A further aspect of the invention provides a retrofit kit for adapting a mass spectrometer to provide a system or a mass spectrometer as described above. The kit may comprise a program element as described above and/or a computer readable medium as described above.

Embodiments of the invention will now be described by way of example only and not in any limitative sense with reference to the accompanying drawings in which:

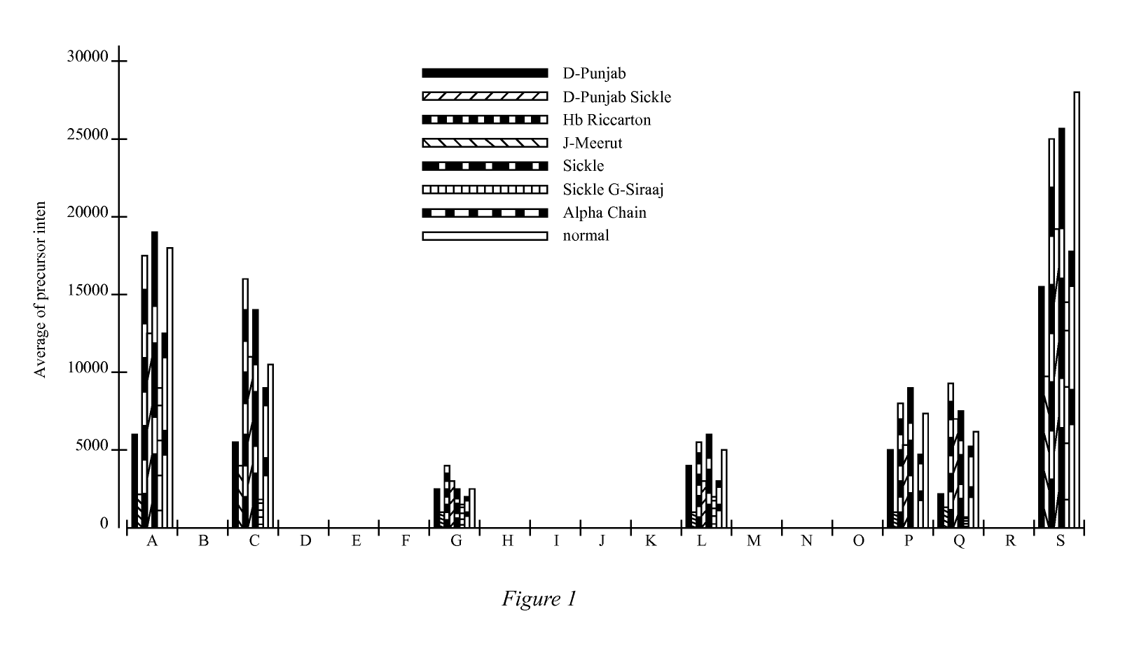

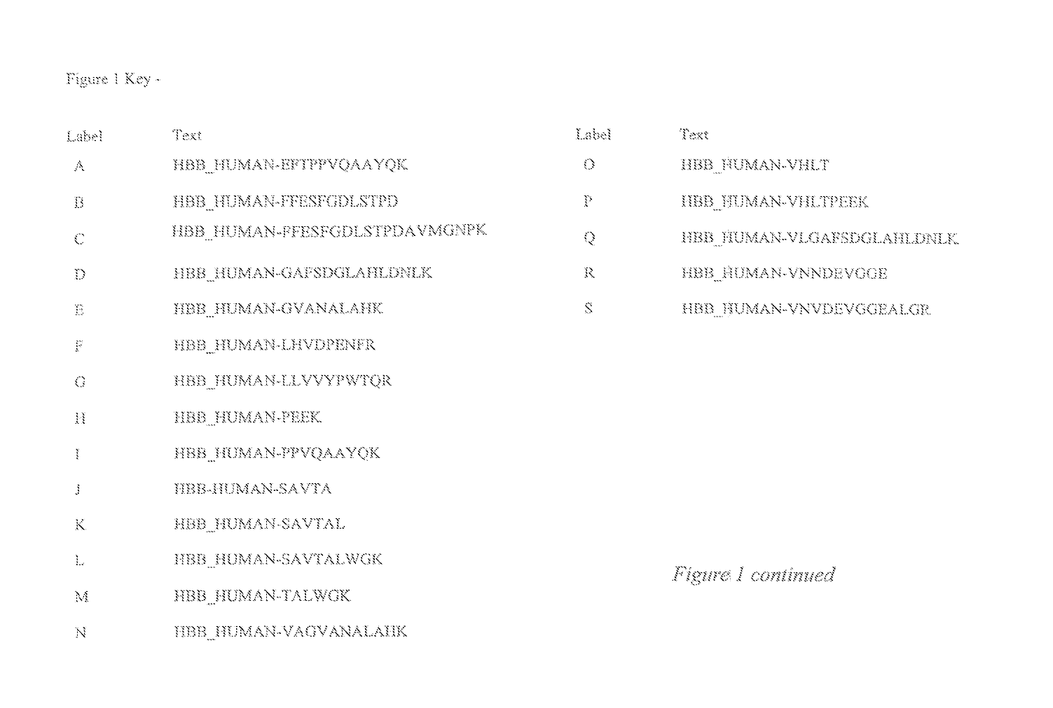

FIG. 1 is a graphical representation of raw, pre-normalization, peptide intensity distributions for Hemoglobin B and;

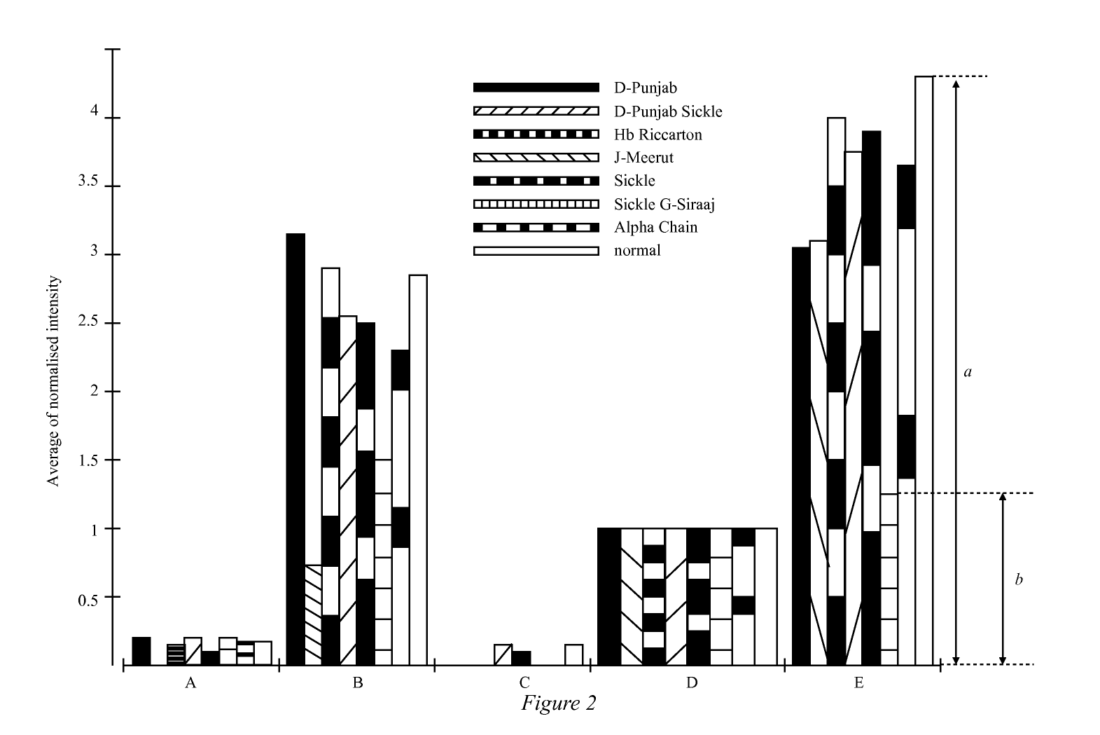

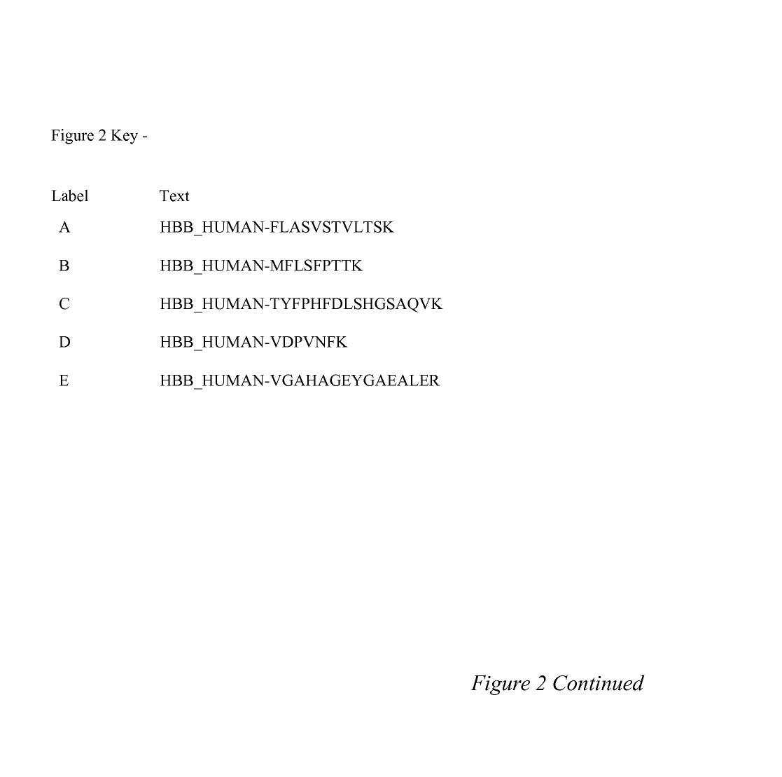

FIG. 2 is a graphical representation of Normalized peptide intensity distributions of Hemoglobin A.

Protein concentration determination in blood is routinely applied in clinics and hospitals to assess patient health status. Abnormalities, that is, an increased or decreased protein concentration versus the norm can be indicative for a disorder or disease. As an example, one of the many tests undertaken on blood samples in screening laboratories is the determination of hemoglobin (Hb) concentration, Detection of anaemia is a common reason for the test. In antenatal clinics, an abnormally low level of Hb from such an analysis of the blood from a potential parent would indicate anaemia and result in further investigation of the cause, particularly to ascertain the risk to the unborn child.

Embodiments of the present invention will now be described with respect to the specific application of this method for the analysis of Hemoglobin in the blood, however, it would be clear to a person skilled in the art that the above invention would be suitable for the analysis of any protein and/or peptide based analyte of interest within a biological sample.

Examples of other analytes of interest that may be analyzed according to the invention include, but are not limited to Lactose dehydrogenase, Malate dehydrogenase, phosphoglandin dehydrogenase, Esterase, Transferrin, Albumin, Phosphoglucomutase, Acid phosphatase, Superoxide dismutase and Glutamic-pyruvic transaminase.

Measurement of Hb concentration in whole blood using a MS-based approach and results indicate that the proposed method shows very good correlation with current hospital measurements using known techniques

Hospitals typically use a haematology automated analyser to measure the concentration of Hb in Blood (For example the SYSMEX XE-2100). Here Hb determination is achieved using the sodium lauryl sulfate (SLS)-hemoglobin method and fluoresecent flow cytometry. Note that this method cannot determine the concentration of individual variants but only provide a total concentration value.

A number of blood samples were submitted for analysis by ESI-MS to measure the correlation between the total Hb concentration as measured by the clinical assay and the MS based procedure. The MS based approach used for measuring the total Hb concentration of each sample required a known quantity of digested ADH/Enolase be spiked into the Hb digest solution as an internal calibrant. The average intensity of the three most intense tryptic peptides may be automatically calculated for the Hb and ADH/Enolase during the data processing. The average MS signal response from the ADH or Enolase is then used to determine a universal signal response factor for the sample (counts/mol of protein). This value is then applied to determine the absolute concentration of the Hb isoforms to get a total value for the concentration of Hb the sample.

Blood samples were obtained from both male and female adults whose normal Hb levels are 12.5-17.0 g/dL and 11.4-15.0 g/dL, respectively. Preliminary data from the LC-MS approach, performed on both TDC and ADC detector platforms, shows excellent correlation with the clinical measurements and a coefficient of variance <10% is routinely attained.

One embodiment of the invention relates to a method of identifying any variants present for an analyte of interest in a sample. However, before this embodiment of the invention can be performed, identification of candidate peptides that will indicate the presence and quantity of each variant for the sample of interest should be performed.

In one aspect of the invention, candidate peptides that will indicate the presence and quantity of each variant for the sample of interest are identified by the following means.

Whole blood samples were diluted 50-fold with water and digested with trypsin under denaturing conditions. For injection onto a nanoscale LC system, samples were diluted with water and known concentration of a protein internal standard (in 0.1% formic acid).

Nanoscale LC separations were performed on a microfluidic nanotile (TRIZAIC), with 2-minutes sample loading and trapping prior to separation on the analytical column at 450 nL/min. The nanotile emitter was positioned close to the orifice of an oa-ToF MS and this was operated in a data independent scanning mode, whereby alternate scans of low and elevated collision energy provided information about intact peptides and their associated fragment ions, respectively.

Data were processed, searched and Hb on-column amounts calculated relative to the concentration of a digested protein which was subsequently used as the internal standard.

The protein on column concentrations were estimated as described by Silva et al. Briefly, the average ion intensity of the three most abundant peptides identified to a protein is standardized to that of an internal standard spiked into the sample at known concentration. However, the observed signal intensity of sequence common peptides can be a summed value arising from redundant identifications. This is advantageous from a qualitative perspective since the intensity of the redundant peptides is cumulative. From a quantitative perspective, it hampers data analysis, especially if the contribution of the individual protein isoform cannot be addressed or accessed. An extension to the earlier presented absolute quantification schema was utilized. Namely, the average intensity is calculated for the proteotypic peptides of every isoform or homolog. These intensities peptides are subsequently used to segment the total observed intensity of the common peptide belonging to each parent protein. In instances where no proteotypic peptide signals can he identified or detected, the identified proteins will be grouped and an absolute amount assigned to the group as a whole. Next, the peptides are re-ordered based on their segmented intensities for the sequence common and non-segmented intensities of the proteotypic peptides and the molar amounts calculated.

Normal alpha and beta hemoglobin subunit amounts are determined as described by Silva et al. Natural variants can skew/underestimated the total hemoglobin concentration determination results, dependent on concentration of the variant(s) and the contribution of the observed peptide intensities of the variant(s) to the peptide intensities of the alpha and beta hemoglobin subunits. The relative concentration of the variant(s) can be estimated and used as a correction factor for the total hemoglobin concentration determination and is a theme variation on the isoform/homology filtering described above. The following logic was applied:
I. The variant(s) will cause disconnect (s) in the normal peptide intensity distribution of either the alpha or beta variant. Of note, disconnects and their magnitudes are variant and variant concentration dependent.
II. One or more peptides are selected for data normalization. Their selection is based on distribution consistency between samples see FIG. 1 where two of the beta hemoglobin peptides can be possibly used for normalization. The highlighted peptides illustrate distribution consistency and are candidates for intensity normalization.
III. The intensities are scaled to the peptide(s) of interest and re-plotted—see FIG. 2 where TYFPHFDLSHGSAQVK of alpha hemoglobin was selected for normalization. The highlighted peptide illustrates the peptide selected for normalization.
IV. Peptide(s) are identified that can be used for correction—in the case of D-Punjab/Sickle in FIG. 2, MFLSFPTTK could be selected and for Sickle/G-Siraaj, MFLSFPTTK and VGAHAGEYGAEALER are candidate peptides for correction.
V. The correction factor is expressed as the b/a—see FIG. 2—averaged out for all possible corrections for a given variant.

It would be appreciated by a person skilled in the art that there may be several different independent candidates that may be useful for the identification of specific variants.

It would be appreciated by a person skilled in the art that there may be many different substances other than ADH or Enolase that may be used as an internal calibrant for the analysis

It would be apparent to a person skilled in the art that upon identification of the candidate peptides for each variant of the substance of interest, it should be possible to search for this candidate peptide in future searches to identify the variant in further samples without the need to proceed with all the steps to identify the candidate peptide for each sample.

In this embodiment, the proposed method would provide quantitative—both relative and absolute—and qualitative information for the normal and variant proteins within a single experiment.

The qualitative aspect relies on the identification of the peptides of interest post proteolytic digestion and analysis by LCMS. The concentration determination of the normal is achieved by the method described by Silva et al. The contribution of the variant(s) to the total haemoglobin concentrations is identified in the present invention. From this information, the relative amount of the variant(s) can also be derived.

In the preferred embodiment, one aspect of the invention relates to a method of analysis of analytes of interest and variants of those analytes. This method may contain the following steps:—
Allow sample denaturation in appropriate denaturing conditions
Provide a suitable digest for the sample to allow digestion of the protein.
Spike the sample with a known amount of a protein based calibrant
Performing LCMS or LCMSMS analysis on the spiked sample
Confirm presence/absence of any variants by the study of the candidates identified in the previous experimental description
determine amount and concentration of normal for of the analyte of interest within the sample using the known amount of calibrant
determine amount and concentration variants of the analyte of interest within the sample using the known amount of analyte of interest and known proportion of variant from the data.

In one embodiment the appropriate denaturing conditions may include addition of a detergent (eg Rapigest) and heating.

It would be apparent to a person skilled in the art that many other ways of treating samples for denaturisation may be used.

In less preferred embodiments, denaturation may not be essential. It may be possible to perform the invention without treating the sample to denaturation.

In one embodiment the digest is a tryptic digest. It would be apparent to a person skilled in the art that many other digests may be used. In less preferred embodiments, digestion may not be essential.

Calibrants should be chosen to avoid any interferences between the calibrant and the sample of interest within the data. In one embodiment this may be chosen from a different species from the sample in question.

In the preferred embodiment the mass spectrometer would be enabled to perform consecutive scans in a high followed by a low fragmentation mode, this may be performed by switching the collision energy from high, to low collision energy as disclosed in U.S. Pat. No. 6,717,130, or by bypassing the collision cell when in low fragmentation mode. In the preferred embodiment, the mass spectrometer of interest should be a ‘Quadrupole-OAToF’ geometry Mass Spectrometer.

In the preferred embodiment a software program would study the results from the mass spectrometer to check the candidate sequences to detect any potential variants present.

In a further embodiment Clinical determination of total Hb concentration and HbA2 using an MS-based approach incorporating a microfluidic nanotile is disclosed.

In the present embodiment one of the many tests undertaken on blood samples in hospital screening laboratories is the determination of hemoglobin (Hb) concentration. Detection of anemia is a common reason for the test. For example, in antenatal clinics an abnormally low level of Hb from such an analysis of the blood from a potential parent would indicate anemia and result in further investigation of the cause, particularly to ascertain the risk to the unborn child.

We have undertaken a study to measure Hb concentration in whole blood using an MS-based approach. The method also measures the level of the minor component, Hb A2 (normally ˜3%), an important bio-marker for β-thalassemia trait. The approach shows good correlation (CV<10%) with hospital assays.

Whole blood samples were diluted 50-fold with water and digested with trypsin under denaturing conditions. For injection onto a nanoLC system, samples were diluted with water and known concentration of Yeast ADH in 0.1% formic acid.

Nanoscale LC separations were performed on a microfluidic nanotile, with 2-minutes sample loading and trapping prior to separation on the analytical column at 450 nL/min. The nanotile emitter was positioned close to the orifice of an oa-ToF MS and this was operated in a data independent scanning mode, whereby alternate scans of low and elevated collision energy provided information about intact peptides and their associated fragment ions, respectively.

Data were processed, searched and Hb on-column amounts calculated relative to the concentration of ADH internal standard.

A number (N>20) of blood samples were submitted for analysis by ESI-MS to measure the correlation between the total Hb concentration as measured by the clinical assay and the MS based procedure. In brief, the MS based approach used for measuring the total Hb concentration of each sample required a known quantity of digested ADH be spiked into the Hb digest solution. The average intensity of the three most intense tryptic peptides is automatically calculated for Hb and ADH during the data processing. The average MS signal response from ADH is then used to determine a universal signal response factor (counts/mol of protein). This value is then applied to determine the absolute concentration of the Hb isoforms.

Blood samples were obtained from both male and female adults whose normal Hb levels are 12.5-17.0 g/dL and 11.4-15.0 g/dL, respectively. Preliminary data from the LC-MS approach, performed on both TDC and ADC detector platforms, shows excellent correlation with the clinical measurements and a coefficient of variance <10% is routinely attained.

Furthermore, we applied the MS approach to the measurement of the δ/(δ+β) globin peptide ratios as potential surrogate markers of HbA2, a biomarker used in population screening for β-thalassemia trait. We observed excellent correlation for this measurement between the ESI-MS analysis and the hospital cation-exchange LC method.

It will be appreciated by those skilled in the art that any number of combinations of the aforementioned features and/or those shown in the appended drawings provide clear advantages over the prior art and are therefore within the scope of the invention described herein.

1. A method for the detection and quantitation of analytes of interest and variants of the analyte of interest comprising the steps of:
(i) providing a sample containing an analyte of interest;
(ii) spiking sample with known amount of calibrant;
(iii) performing an LCMS or LCMSMS analysis on the spiked sample to produce a data set;
(iv) determining from said data set the relative quantity of analyte of interest to calibrant;
(v) calculating the absolute quantity of the analyte of interest from said relative quantity of analyte of interest and said known amount of calibrant;
(vi) searching for one or more previously identified candidate sequences for one or more known variants denoted by one or more specific peaks to identify the presence of said one or more known variants within the sample;
(vii) determining the relative quantity or amount of said one or more variants to said analyte of interest; and
(viii) calculating the absolute quantity or amount of any of said one or more variants present in the sample.

2. Method according to claim 1 further comprising identifying the candidate sequences prior to the searching step by:
(i) spiking a sample with a known amount of calibrant;
(ii) performing an LCMS or LCMSMS analysis on the spiked sample to produce a candidate data set;
(iii) selecting from the candidate data set one or more sequences for data normalisation;
(iv) scaling the sample intensities to sequences of interest; and
(v) identifying one or more candidate sequences that can be used for correction,

3. A method for the identification of candidate sequences for one or more known variants of an analyte, the method comprising the steps of:
(i) spiking a sample with a known amount of calibrant;
(ii) performing an LCMS or LCMSMS analysis on the spiked sample to produce a candidate data set;
(iii) selecting from the candidate data set one or more sequences for data normalisation;
(iv) scaling the sample intensities to sequences of interest; and
(v) identifying one or more candidate sequences that can be used for correction,

4. (canceled)

5. (canceled)

6. Method according to claim 3, wherein a digest is added to said sample.

7. Method according to claim 3, wherein denaturation of said sample is performed.

8. Method according to claim 3, wherein the analyte of interest is hemoglobin.

9. A system for carrying out a method according to claim 1, the system comprising: a mass spectrometer for producing at least one measured spectrum of data from a sample and a processor configured or programmed or adapted to carry out a method according to claim 1.

10. A system according to claim 9 further comprising a memory means for storing a library of candidate sequences.

11. (canceled)

12. (canceled)

13. (canceled)

14. A mass spectrometer specifically adapted to carry out a method according to claim 1.

15. A retrofit kit for adapting a mass spectrometer to provide a system according to claim 9.

16. A system according to claim 9, wherein the mass spectrometer has a Quadrupole OAToF geometry.

17. A system according to claim 9, wherein the mass spectrometer is arranged to switch between a high and a low fragmentation mode.

18. Method according to claim 1, wherein a digest is added to said sample.

19. Method according to claim 1, wherein denaturation of said sample is performed.

20. Method according to claim 1, wherein the analyte of interest is hemoglobin.

21. A mass spectrometer specifically adapted to carry out a method according to claim 3.

22. A system for carrying out a method according to claim 3, the system comprising: a mass spectrometer for producing at least one measured spectrum of data from a sample and a processor configured or programmed or adapted to carry out a method according to claim 3.

23. A system according to claim 22 further comprising a memory means for storing a library of candidate sequences.

24. A system according to claim 22, wherein the mass spectrometer has a Quadrupole OAToF geometry.

25. A system according to claim 22, wherein the mass spectrometer is arranged to switch between a high and a low fragmentation mode
